(19) United States
(12) Patent Application Publication
(10) Pub. No.: US 2022/0285030 A1
(43) Pub. Date: Sep. 8, 2022

(54) INFORMATION PROCESSING METHOD

(71) Applicant: NEC Corporation, Minato-ku, Tokyo (JP)

(72) Inventors: Yuki KOSAKA, Tokyo (JP); Toshinori HOSOI, Tokyo (JP); Masahiro KUBO, Tokyo (JP)

(73) Assignee: NEC Corporation, Minato-ku, Tokyo (JP)

(21) Appl. No.: 17/632,871

(22) PCT Filed: Aug. 21, 2019
(86) PCT No.: PCT/JP2019/032582

§ 371 Date: Feb. 4, 2022

(51) Int. Cl. G16H 50/30

(52) U.S. Cl. CPC G16H 50/30; G16H 20/30

(57) ABSTRACT

An information processing apparatus according to the present invention includes an input unit and a generating unit. The input unit accepts input of a first assessment value representing assessment of a subject at a predetermined moment and a second assessment value representing assessment of the subject after a lapse of a predetermined time from the predetermined moment for each of a plurality of items set in FIM (Functional Independence Measure). The generating unit generates a model for calculating the second assessment value with respect to the first assessment value for each of the plurality of items of the FIM based on information representing an association between the items of the FIM.

### TECHNICAL FIELD

The present invention relates to an information processing method, an information processing apparatus, and a program.

### BACKGROUND ART

Injuries, illnesses, aging and so on may reduce a function of activities of daily living and a cognition function. In such cases, rehabilitation is performed in a rehabilitation facility for recovery of the function of activities of daily living and the cognition function. Then, the rehabilitation facility needs to grasp the conditions of a motor function related to activities of daily living and a cognition function of a patient subject to rehabilitation and, as an example of an index for measuring such states of the patient, the FIM (Functional Independence Measure: an index for measuring a motor function related to activities of daily living and a cognition function) is used. For example, as shown in Patent Document 1, the FIM is composed of a total of eighteen items including thirteen kinds of motor items and five kinds of cognition items, and each of the items is assessed by a degree of need for assistance of a four-level or seven-level scale.

Then, the rehabilitation facility needs to predict the recovery of a patient in order to develop a rehabilitation plan for the patient and give information about future assistance to the patient and the patient's family. For this, it is considered to predict the assessment of each item of the FIM from the current situation of a new patient by referring to a case showing the outcome of rehabilitation of a past patient, for example. The above FIM is an example as an index for measuring the condition of the body of a human as a patient, and it is also possible to predict the assessment of items set in another index for assessing the condition of a human body different from the FIM.
Patent Document 1: Japanese Unexamined Patent Application Publication No. JP-A 2017-027476

However, since the FIM is composed of eighteen items as mentioned above, it is difficult to accurately predict the assessments of all the items.

### SUMMARY

Accordingly, an object of the present invention is to provide an information processing method, an information processing apparatus and a program that can solve the abovementioned problem of difficulty in accurate prediction of the assessments of all the items of the FIM.

An information processing method as an aspect of the present invention includes: accepting input of a first assessment value representing assessment of a subject at a predetermined moment and a second assessment value representing assessment of the subject after a lapse of a predetermined time from the predetermined moment for each of a plurality of items set in FIM (Functional Independence Measure); and generating a model for calculating the second assessment value with respect to the first assessment value for each of the plurality of items of the FIM based on information representing an association between the items of the FIM.

Further, an information processing method as an aspect of the present invention includes inputting, into a model generated to calculate a second assessment value representing assessment of a subject after a lapse of a predetermined time from a predetermined moment with respect to a first assessment value representing assessment of the subject at the predetermined moment for each of a plurality of items set in FIM (Functional Independence Measure) based on information representing an association between the items of the FIM, a new first assessment value for each of the plurality of items of the FIM, and outputting a value calculated with the model in accordance with the input of the new first assessment value.

Further, an information processing apparatus as an aspect of the present invention includes: an input unit configured to accept input of a first assessment value representing assessment of a subject at a predetermined moment and a second assessment value representing assessment of the subject after a lapse of a predetermined time from the predetermined moment for each of a plurality of items set in FIM (Functional Independence Measure); and a generating unit configured to generate a model for calculating the second assessment value with respect to the first assessment value for each of the plurality of items of the FIM based on information representing an association between the items of the FIM.

Further, an information processing apparatus as an aspect of the present invention includes: an input unit configured to input, into a model generated to calculate a second assessment value representing assessment of a subject after a lapse of a predetermined time from a predetermined moment with respect to a first assessment value representing assessment of the subject at the predetermined moment for each of a plurality of items set in FIM (Functional Independence Measure) based on information representing an association between the items of the FIM, a new first assessment value for each of the plurality of items of the FIM; and a predicting unit configured to output a value calculated with the model in accordance with the input of the new first assessment value.

Further, a computer program as an aspect of the present invention includes instructions for causing an information processing apparatus to realize: an input unit configured to accept input of a first assessment value representing assessment of a subject at a predetermined moment and a second assessment value representing assessment of the subject after a lapse of a predetermined time from the predetermined moment for each of a plurality of items set in FIM (Functional Independence Measure); and a generating unit configured to generate a model for calculating the second assessment value with respect to the first assessment value for each of the plurality of items of the FIM based on information representing an association between the items of the FIM.

Further, a computer program as an aspect of the present invention includes instructions for causing an information processing apparatus to realize: an input unit configured to input, into a model generated to calculate a second assessment value representing assessment of a subject after a lapse of a predetermined time from a predetermined moment with respect to a first assessment value representing assessment of the subject at the predetermined moment for each of a plurality of items set in FIM (Functional Independence Measure) based on information representing an association between the items of the FIM, a new first assessment value for each of the plurality of items of the FIM; and a predicting unit configured to output a value calculated with the model in accordance with the input of the new first assessment value.

Further, an information processing method as an aspect of the present invention includes: accepting input of a first assessment value representing assessment of a subject at a predetermined moment and a second assessment value representing assessment of the subject after a lapse of a predetermined time from the predetermined moment for each of a plurality of items set in a predetermined index for assessing a condition of a human body; and generating a model for calculating the second assessment value with respect to the first assessment value for each of the plurality of items of the predetermined index based on information representing an association between the items of the predetermined index.

Further, an information processing method as an aspect of the present invention includes inputting, into a model generated to calculate a second assessment value representing assessment of a subject after a lapse of a predetermined time from a predetermined moment with respect to a first assessment value representing assessment of the subject at the predetermined moment for each of a plurality of items set in a predetermined index for assessing a condition of a human body based on information representing an association between the items of the predetermined index, a new first assessment value for each of the plurality of items of the predetermined index, and outputting a value calculated with the model in accordance with the input of the new first assessment value.

With the configurations as described above, the present invention enables accurate prediction of the assessments of all the items of the FIM.

### BRIEF DESCRIPTION OF DRAWINGS

FIG. 1 is a view for describing the FIM;

FIG. 2 is a block diagram showing a configuration of an information processing apparatus according to the present invention;

FIG. 3 is a view showing an example of equations used at the time of generating a model in the information processing apparatus disclosed in FIG. 2 in a first example embodiment of the present invention;

FIG. 4 is a view showing an example of data included in the equations disclosed in FIG. 3;

FIG. 5 is a view showing an example of data included in the equations disclosed in FIG. 3;

FIG. 6 is a view showing an example of data included in the equations disclosed in FIG. 3;

FIG. 7 is a flowchart showing an operation of the information processing apparatus disclosed in FIG. 1;

FIG. 8 is a view showing an example of data included in an equation used at the time of generating a model in the information processing apparatus disclosed in FIG. 2 in a second example embodiment of the present invention;

FIG. 9 is a block diagram showing a hardware configuration of an information processing apparatus in a third example embodiment of the present invention;

FIG. 10 is a block diagram showing a configuration of the information processing apparatus in the third example embodiment of the present invention;

FIG. 11 is a block diagram showing another configuration of the information processing apparatus in the third example embodiment of the present invention;

FIG. 12 is a flowchart showing an operation of the information processing apparatus in the third example embodiment of the present invention; and

FIG. 13 is a flowchart showing another operation of the information processing apparatus in the third example embodiment of the present invention.

### EXAMPLE EMBODIMENTS

### First Example Embodiment

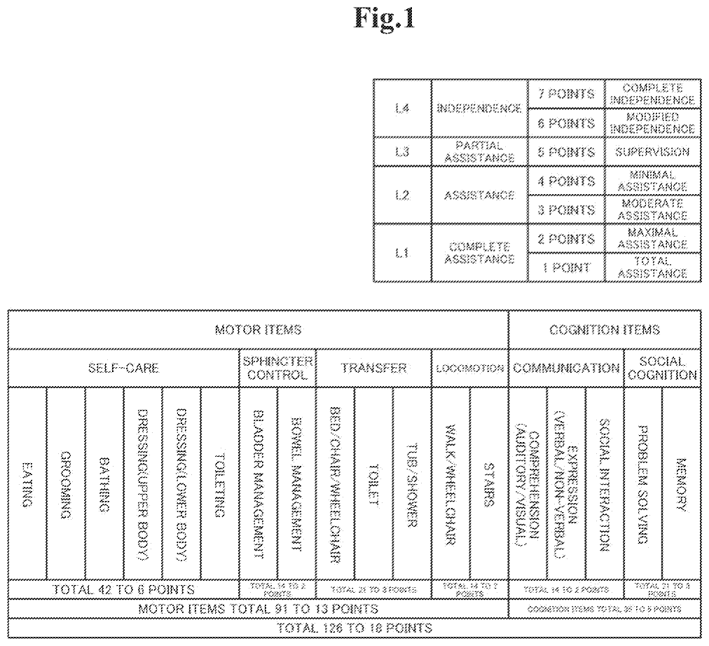

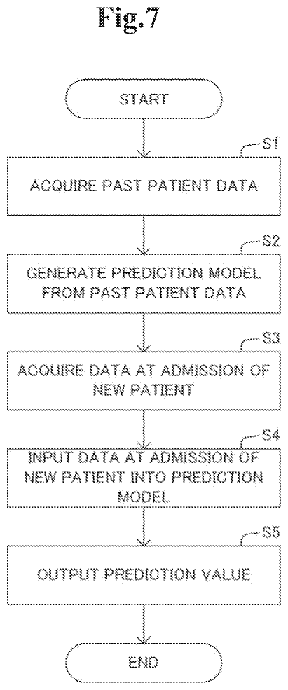

A first example embodiment of the present invention will be described with reference to FIGS. 1 to 7. FIGS. 1 to 6 are views for describing a configuration of an information processing apparatus, and FIG. 7 is a view for describing a processing operation of the information processing apparatus.

### [Configuration]

An information processing apparatus 10 according to the present invention is used for, when a patient (a subject) whose function of activities of daily living and cognition function have deteriorated due to injury, illness, aging and so on is rehabilitated in a rehabilitation facility for recovery of the function of activities of daily living and the cognition function, predicting the future condition of the patient. Patients to be subject to rehabilitation include a patient with a cerebrovascular disease such as cerebral infarction or cerebral hemorrhage, but a patient in any condition may be the subject. To be specific, the information processing apparatus 10 is used for, by using the FIM (Functional Independence Measure) that is an index for measuring a motor function related to activities of daily living and a cognition function of a patient, predicting the assessment value of each item of the FIM at the time of future discharge (after a lapse of a predetermined time from the time of admission) from information of the patient including the assessment value of each item of the FIM at the time of admission (a predetermined time). By thus predicting the assessment value of each item of the FIM at the time of discharge of the patient, the facility can develop an efficient rehabilitation plan for the patient. Moreover, the facility can provide appropriate information about future assistance for the patient and the patient's family based on the result of the prediction.

The time of admission stated above is not necessarily limited to the day of admission, and may be a time that can be substantially regarded as the time of admission, such as a time when the assessment of each item of the FIM is performed several days after the day of admission. Moreover, the time of discharge stated above is not necessarily limited to the day of discharge, and may be a day when discharge is scheduled or a time when a preset period such as two weeks or one month has elapsed from the time of admission. Furthermore, the time of admission and the time of discharge stated above are examples, and the information processing apparatus 10 may predict, based on the condition of the patient at any moment during hospitalization, the assessment value of each item of the FIM at any later moment.

Here, the FIM that is an index for measuring a motor function related to activities of daily living and a cognition function of a patient will be described with reference to FIG. 1. As shown in FIG. 1, the FIM is composed of a total of eighteen items including thirteen kinds of motor items for assessing the “motor function” of a patient and five kinds of cognition items for assessing the “cognition function” of a patient. To be specific, the FIM includes, as the abovementioned motor items, items for assessing the patient's function of activities of a “self-care” category such as “eating”, “glooming”, “bathing”, “dressing (upper body)”, “dressing (lower body)” and “toileting”, items for assessing the patient's function of activities of a “sphincter control” category such as “bladder management” and “bowel management”, items for assessing the patient's function of activities of a “transfer” category such as “bed/chair/wheelchair”, “toilet” and “tub/shower”, and items for assessing the patient's function of activities of a “locomotion” category such as “walk/wheelchair” and “stairs”. Moreover, the FIM includes, as the abovementioned cognition items, items for assessing the patient's function of a “communication” category such as “comprehension (auditory/visual)” and “expression (verbal/non-vernal), and items for assessing the patient's function of a “social cognition” category such as “social interaction”, “problem solving” and “memory”.

With the FIM, a degree of assistance necessary for a patient is assessed on a four-level or seven-level scale for each of the abovementioned items. For example, as shown in the upper right part of FIG. 1, each item may be assessed by degrees of four-level scale such as “L1: complete dependence on helper”, “L2: helper”, “L3: modified dependence on helper”, and “L4: no helper”. Moreover, for example, each item may be assessed by degrees of seven-level scale such as “total assistance”, “maximal assistance”, “moderate assistance”, “minimal assistance, “supervision”, “modified independence”, and “complete independence”. In the case of such assessment on a seven-level scale, a patient may be assessed by aggregating levels given to the respective assessment degrees for each item, each category, and each function.

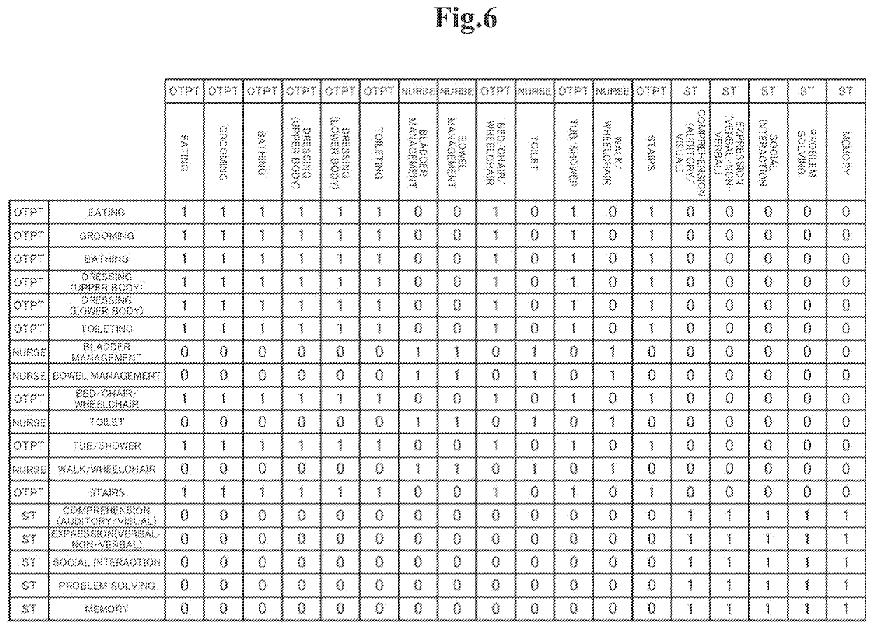

The assessment of each item of the FIM described above is generally performed by a specialist assisting a patient as an assessor. For example, the items such as “eating”, “grooming”, “bathing”, “dressing (upper body)”, “dressing (lower body)”, “toileting”, “bed/chair/wheelchair”, “tub/shower” and “stairs” are assessed by an “occupational therapist (OP)” or a “physical therapist (PT)”, which will be described later with reference to FIG. 6. Moreover, the items such as “bladder management”, “bowel management”, “toilet” and “walk/wheelchair” are assessed by a “nurse”. Moreover, the items such as “comprehension (auditory/visual)”, “expression (verbal/non-verbal)”, “social interaction”, “problem solving” and “memory” are assessed by a “speech-hearing therapist (ST).

The assessment value of each item of the above FIM is input into a data management apparatus 20 by the specialist serving as an assessor mentioned above and stored as patient data. For example, in the data management apparatus 20, patient data of each patient is stored as an electronic patient chart. In the electronic patient chart, information such as “gender”, “age group”, “consciousness level (JCS: Japan Coma Scale)”, “disease name” “paralysis condition” “assessment value of each item of FIM at admission (first assessment value)” and “assessment value of each item of FIM at discharge (second assessment value)” are stored as the patient data, for example. However, the patient data is not necessarily limited to including the information of the contents mentioned above, and may include only part of the abovementioned information or may include other information. The patient data of a patient who is still hospitalized does not include “assessment value of each item of FIM at discharge”.

According to the present invention, the information processing apparatus 10 predicts the assessment value of each item of the FIM at the time of discharge of an initially or just admitted patient by using the patient data stored in the data management apparatus 20 as described above. Therefore, the information processing apparatus 10 includes the following configuration in order to realize functions to perform a process to generate a model for predicting the assessment value of each item of the FIM at the time of discharge of a patient and a process to predict the assessment value of each item of the FIM at the time of discharge of a patient by using the model.

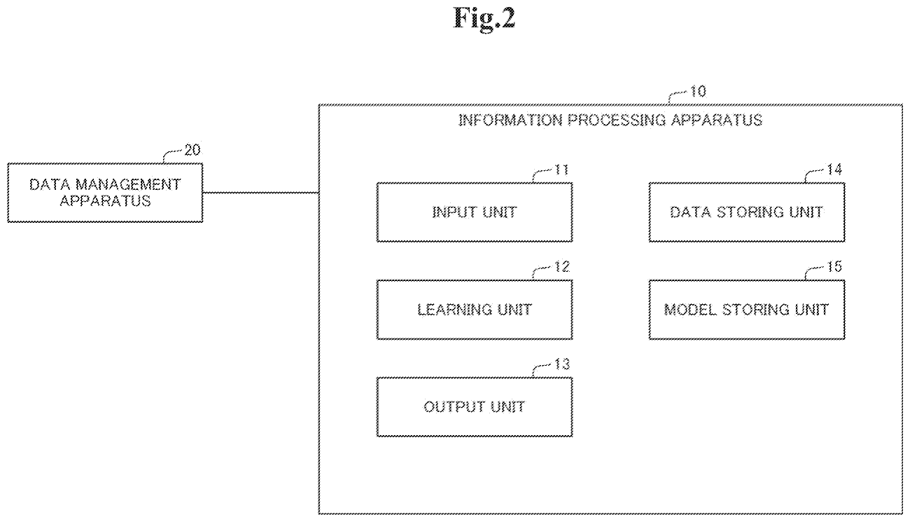

First, the information processing apparatus 10 is composed of one or a plurality of information processing apparatuses each including an arithmetic logic unit and a storage unit. The information processing apparatus 10 includes an input unit 11, a learning unit 12 and an output unit 13 that are structured by a program executed by the arithmetic logic unit as shown in FIG. 2. The information processing apparatus 10 also includes a data storing unit 14 and a model storing unit 15 that are formed in the storage unit. The respective components will be described in detail below.

The input unit 11 requests patient data from the data management apparatus 20, accepts input of the patient data, and stores into the data storing unit 14. In the model generation process, the input unit 11 requests and acquires patient data of an already discharged patient as learning data. For example, the input unit 11 requests patient data in which a flag representing that a patient has been discharged is set and patient data in which the assessment value of each item of the FIM at the time of discharge has been input, and acquires as learning data.

Further, in the prediction process, the input unit 11 requests and acquires patient data of a patient subject to the prediction process who has not been discharged yet as prediction data. For example, the input unit 11 requests patient data in which a flag representing that a patient has been discharged is not set and patient data in which the assessment value of each item of the FIM at the time of discharge has not been input, and acquires as prediction data. The patient data as prediction data of a patient subject to the prediction process is acquired after a model is generated as will be described later, but may be acquired at any timing.

The learning unit 12 (generating unit) performs machine learning by using patient data acquired as learning data stated above, generates a model for predicting the assessment value of each item of the FIM at the time of discharge of a patient, and stores the model into the model storing unit 15. At this time, the learning unit 12 generates, by machine learning, a model function represented by a function (f_i(X_n)) where “basic information” such as “gender”, “age group”, “consciousness level”, “disease name” and “paralysis condition” in patient data and “information at admission” such as “assessment value of each item of the FIM at admission (first assessment value)” are an input value (X_n: n=1, . . . , N (N: number of patients)) and “assessment value of each item of the FIM at discharge (second assessment value)” is an output value (y_i: i=1, . . . , 18 (items)). That is to say, the learning unit 12 generates a model function for calculating an output value (y_i) to an input value (X_n). In this example, assuming y_i={1, 2, 3, 4, 5, 6, 7}, the assessment value of each item of the FIM is assessed on the seven-level scale described above.

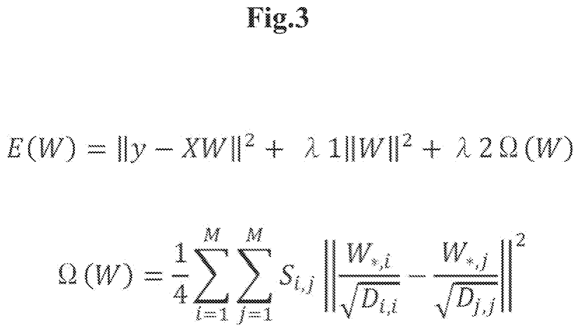

The learning unit 12 generates a model function f_i by using Ridge regression in this example embodiment. To be specific, the learning unit 12 calculates a parameter (W) (coefficient) of each term constituting a model function (f_i) so as to minimize an assessment function (loss function) shown in the upper part of FIG. 3, thereby generating the model function (f_i).

In this example embodiment, an assessment function including two regularization terms each including a parameter (W) is used as shown in the upper part of FIG. 3. To be specific, the first regularization term is “λ1∥w∥²” and the second regularization term is “λ2Ω(W)”. At this time, λ1 and λ2 are parameters that adjust the degrees of influence given by the respective regularization terms to the loss function. The parameters are given beforehand. As λ1 and λ2 have larger magnitudes, they given more influences to the loss function.

In this example embodiment, specifically, “Ω(W)” constituting the regularization term of the final term includes an adjacency matrix represented by “Si,j” as shown in the lower part of FIG. 3. The adjacency matrix Si,j is information representing the association between the items of the FIM. For example, “1” is set to between items associated with each other and “0” is set to between items not associated with each other.

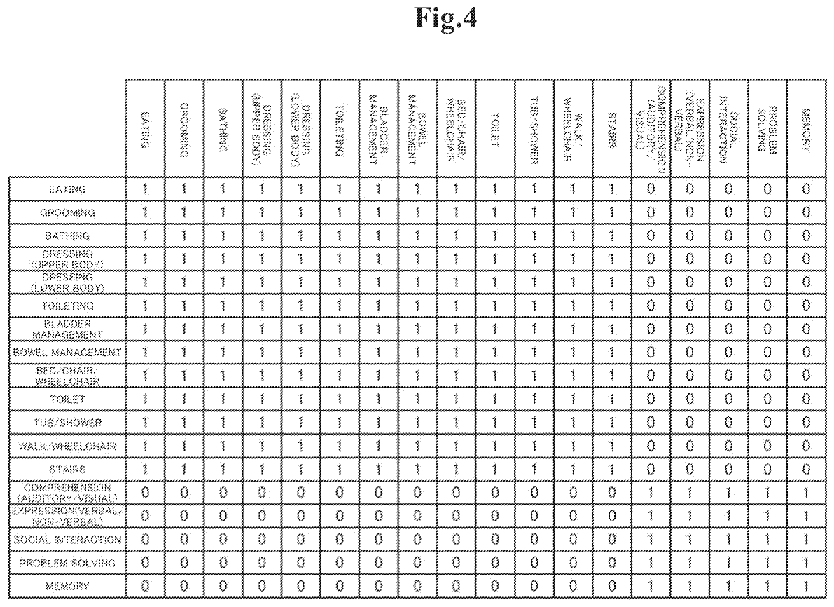

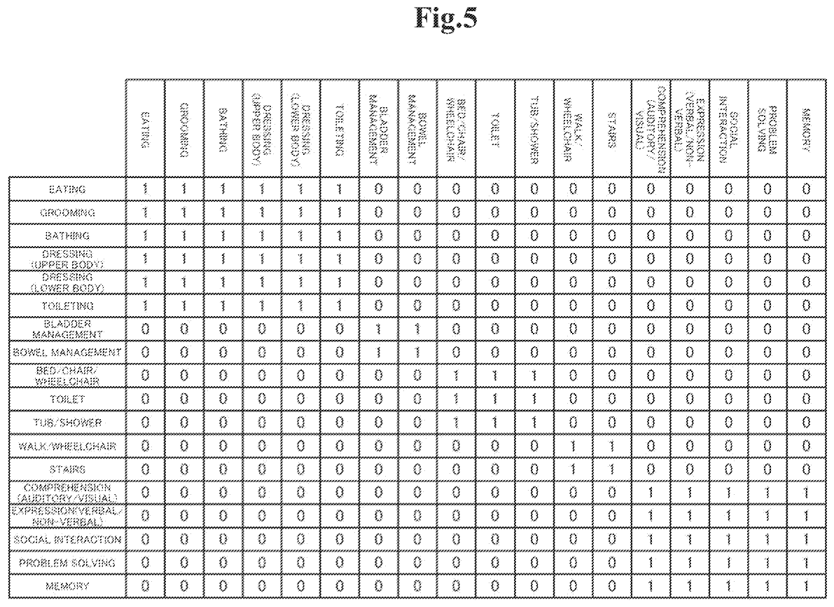

Here, examples of the adjacency matrix Si,j will be described with reference to FIGS. 4 to 6. In the examples of FIGS. 4 and 5, the adjacency matrix Si,j is set based on the similarity of the assessment contents of the respective items of the FIM. To be specific, in the example of FIG. 4, it is assumed that items are associated with each other when “functions” (“motor” or “cognition”) which the items belong to in the FIM shown in FIG. 1 are the same, and “1” is set to between items belonging to the “motor” function and to between items belonging to the “cognition” function. Moreover, in the example of FIG. 5, it is assumed that items are associated with each other when “categories” obtained by further classifying the “functions” described above are the same. To be specific, “1” is set to between items belonging to the same category such as a “self-care” category, a “sphincter control” category, a “transfer” category, a “locomotion” category, a “communication” category, and a “social cognition” category as shown in FIG. 1. Moreover, in the example of FIG. 6, the adjacency matrix Si,j is set based on the “assessor” of each item of the FIM. For example, the assessors of the respective items are written in the outermost rows and columns in FIG. 6. It is assumed that items with the same assessors (“occupational therapist (OP)”, “physical therapist (PT)”, “nurse”, and “speech-hearing therapist (ST)”) are associated with each other, and “1” is set to between the items.

In this example embodiment, by providing a regularization term including an adjacency matrix according to the association between items of the FIM as described above, it is possible to generate a function (f_i) corresponding to the items of the FIM associated with each other so that parameters included in the function (f_i) are similar to each other. That is to say, in the equation shown in the lower part of FIG. 3, the difference between the parameters of a function corresponding to the items of the FIM associated with each other is squared, and the parameters are optimized so as to be similar to each other in order to make the value of an assessment function small.

As described above, regularization using an adjacency matrix is described in the following document and is an existing technique, so that a detailed description thereof will be omitted.

Nozomi Nori, Hisashi Kashima, Kazuto Yamashita, Hiroshi Ikai, and Yuichi Imanaka, “Simultaneous Modeling of Multiple Diseases for Mortality Prediction in Acute Hospital Care” in Proceedings of the 21th ACM SIGKDD International Conference on Knowledge Discovery and Data Mining, pp. 855-864, 2015

The output unit 13 (predicting unit) inputs patient data of a patient who has not been discharged yet acquired as prediction data by the input unit 11 into the model function (f_i) generated as described above. That is to say, the output nit 13 inputs “basic information” such as “gender”, “age group”, “consciousness level”, “disease name” and “paralysis condition” and “information at admission” such as “assessment value of each item of FIM at admission (first assessment value)” as an input value (X_n′) into the model function, and calculates an output value (y_i′) by the model function (f_i(X_n′)). Thus, it is possible to predict the assessment value (for example, a value of seven-level scale) of each item of the FIM at the time of discharge of a patient who is just admitted.

### [Operation]

Next, an operation of the information processing apparatus 10 described above will be descried with reference to a flowchart of FIG. 7. First, the information processing apparatus 10 performs a model generation process to generate a model for predicting the assessment value of each item of the FIM at the time of discharge of a patient. For this, the information processing apparatus 10 requests past patient data from the data management apparatus 20, and acquires the patient data as learning data (step S1).

Then, the information processing apparatus 10 generates, by machine learning, a model function represented by a function where “basic information” including “gender”, “age group”, “consciousness level”, “disease name” and “paralysis condition” and “information at discharge” including “assessment value of each item of FIM at admission” in the patient data input values and “assessment value of each item of FIM at discharge” is an output value (step S2). At this time, the information processing apparatus 10 generates the model function by using Ridge regression, and specifically, optimizes a parameter of each term constituting the model function by using an assessment function in which a regularization term including an adjacency matrix that is information representing the association between items of the FIM is added as described above. Thus, it is possible to generate a model function such that parameters included in the model function corresponding to the items of the FIM associated with each other are similar to each other.

Subsequently, the information processing apparatus 10 performs a prediction process to predict the assessment value of each item of the FIM at the time of discharge of a patient by using the generated model. For this, the information processing apparatus 10 requests patient data of a newly admitted patient or a patient hospitalized but not discharged from the data management apparatus 20, and acquires the patient data as prediction data (step S3). Since the patient has not been discharged yet, the patient data acquired as prediction data does not include the assessment value of each item of the FIM at the time of discharge.

The information processing apparatus 10 inputs “basic information” including “gender”, “age group”, “consciousness level”, “disease name” and “paralysis condition” and “information at admission” including “assessment value of each item of FIM at admission” in the patient data as input values into the model function (step S4). Then, the information processing apparatus 10 outputs “assessment value of each item of FIM at discharge” calculated by the model function as a prediction value (step S5). With this, it is possible to predict the assessment value of each item of the FIM at the time of discharge of an admitted patient (for example, a value of seven-level scale). Then, the output prediction result can be used, for example, for developing an efficient rehabilitation plan for a patient in a facility and for giving an advice about future assistance for the patient and the patient's family.

As described above, according to the present invention, a model for calculating the assessment value of each item of the FIM is generated in consideration of the association between the items of the FIM from information of a past patient who has been rehabilitated. By thus using the association between the items of the FIM, it is possible to accurately and speedily predict the assessment value of each item of the FIM at the time of discharge even if an assessment index including many items is used.

Although a case of predicting the assessment value of each item of the FIM at the time of discharge from patient data at the time of admission of a patient is illustrated above, the assessment value of each item of the FIM at future moment may be predicted using patient data at any moment during hospitalization.

Further, although the assessment values of the items set in the FIM are used above, the value of an item set in another index for assessing the condition of a human body may be used. For example, there is an index for assessing activities of daily living such as the “Barthel Index” for assessing a total of ten items set from two viewpoints including daily living activity and locomotion activity in accordance of the degree of independence, and the values of items of the index may be used to generate a model as described above and calculate a prediction value.

### Second Example Embodiment

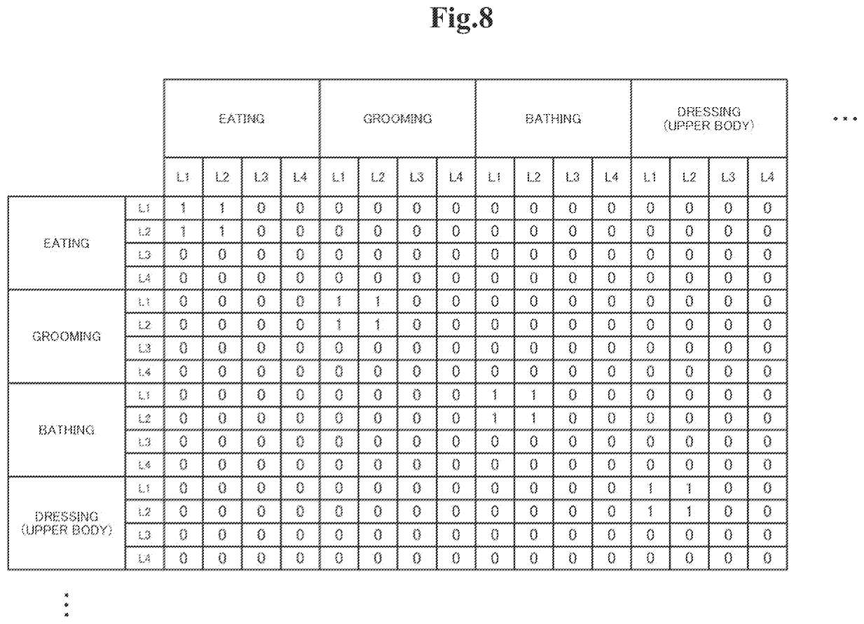

Next, a second example embodiment of the present invention will be described with reference to FIG. 8. FIG. 8 is a view for describing a configuration of an information processing apparatus in the second example embodiment.

The information processing apparatus 10 according to the present invention is used for predicting, from information of a patient including the assessment value of each item of the FIM at the time of admission (a predetermined moment), the assessment value of each item of the FIM at the time of later discharge (after a lapse of a predetermined time from the time of admission) as in the first example embodiment described above. However, this example embodiment is different from the first example embodiment in predicting whether or not the assessment value of each item of the FIM at the time of discharge increases one level or more. A configuration different from that of the first example embodiment will be majorly described below.

First, as in the first example embodiment, the information processing apparatus 10 includes the input unit 11, the learning unit 12 and the output unit 13 that are structured by a program executed by the arithmetic logic unit as shown in FIG. 2. The information processing apparatus 10 also includes the data storing unit 14 and the model storing unit 15 that are formed in the storage unit.

Then, the input unit 11 in this example embodiment requests patient data from the data management apparatus 20, accepts input of the patient data, and stores into the data storing unit 14. In the model generation process, the input unit 11 requests and acquires patient data of a patient who has already been discharged as learning data. In this example embodiment, it is assumed that the assessment values of the respective items of the FIM at the time of admission and at the time of discharge are assessed on a four-level scale including “L1: complete dependence on helper”, “L2: helper”, “L3: modified dependence on helper”, “no helper”. Moreover, in the prediction process, the input unit 11 requests and acquires, as prediction data, patient data of a patient who has not been discharged yet subject to the prediction process.

Then, the learning unit 12 (generating unit) in this example embodiment performs machine learning by using the patient data acquired as the learning data mentioned above, generates a model for predicting whether or not the assessment value of each item of the FIM at the time of discharge of a patient increases at least one level, and stores the model into the model storing unit 15. At this time, the learning unit 12 first sets “basic information” including “gender”, “age group”, “consciousness level”, “disease name” and “paralysis condition” and “information at admission” including “initial value as assessment value of each item of FIM at admission (first assessment value)” in the patient data as an input value (X_n: n=1, . . . , N (N=the number of patients). In this example embodiment, the above “initial value as assessment value of each item of FIM at admission” is a four-level scale assessment value including “L1: complete dependence on helper”, “L2: helper”, “L3: modified dependence on helper” and “L4: no helper”, and the patient data is classified and learned for each item of the FIM and for each initial value.

Further, the learning unit 12 sets, from “assessment value of each item of FIM at admission and at discharge” in the patient data, a value (second assessment value) representing “whether or not assessment value at admission increases one level or more at discharge” in each item of the FIM, as an output value (y_ik: i=1, . . . , 18 (item), k=L1, L2, L3, L4 (initial value)). At this time, the output value y is a binary value such as y={0, 1}, y=1 indicates a case where an initial value of an i^{th} FIM item at the time of discharge increases one level or more from that at the time of admission, and y=0 indicates a case where an initial value of an i^{th} FIM item at the time of discharge does not change or degreases from that at the time of admission. That is to say, as preprocessing of the model generation, the learning unit 12 previously calculates a value (y) representing “whether or not assessment value at admission increases one level or more” from “assessment value of each item of FIM at admission and at discharge”, and sets as an output value. Contrary to the above, the output value may be set as a value indicating “whether or not assessment value at admission decreases one level or more at discharge” in each item of the FIM. That is to say, the output value may be set as a value representing whether or not an assessment value at the time of admission changes at discharge in one direction such as increases or decreases.

Then, the learning unit 12 generates, by machine learning, a model function represented by a function (f_ik(X_n)) that calculates a binary output value as mentioned above with respect to an input value set as described above. At this time, the learning unit 12 generates a model function calculating an output value for each item of the FIM and for each initial value that is the assessment value of each item of the FIM at the time of admission. For example, the learning unit 12 generates, for the FIM item “eating”, a model function corresponding to each of cases where an initial value that is an assessment value at the time of admission of the item “eating” is “L1”, “L2”, and “L3”. That is to say, a model function corresponding to each of the three kinds of initial values is generated for each of the eighteen items, and a total of fifty-four kinds of model functions are collectively generated. In a case where an initial value at the time of admission is “L4: no helper”, it is not necessary to predict a later assessment value, so that a model function is not generated.

In this example embodiment, the learning unit 12 generates a model function (f_ik) by using logistic regression. To be specific, the learning unit 12 generates a model function to calculate a binary output value with respect to an input value by using the classification probability of the sigmoid function. At this time, the learning unit 12 calculates a parameter (W) (coefficient) of each term constituting the model function so as to minimize an assessment function (loss function) as in the first example embodiment, thereby generating the model function. The parameter of each term constituting the model function represents the degree of magnitude of an influence that may be given to an output value by an input value (for example, age, gender, consciousness level, the value of each term of the FIM, and so on) to be input into a variable included by the model function.

Then, in this example embodiment, an assessment function is used in which still another regularization term is added to the two regularization terms including the parameter (W) described in the first example embodiment. To be specific, to the two regularization terms “λ1∥w∥²” and ““λ2Ω(W)” described in the first example embodiment, a third regularization term “λ3Ω′(W)” is added. Here, λ1, λ2, and λ3 are parameters that adjust the degree of influence of each regularization term on the loss function, as described above. This parameter shall be given in advance. As λ1, λ2, and λ3 have larger magnitudes, they have more influence on the loss function.

Here, “Ω′(W)” constituting the third regularization term added in this example embodiment as described above is almost the same as Ω(W) constituting the second regularization term shown in FIG. 3 described in the first embodiment, but the adjacency matrix “Si,j” is different. The adjacency matrix S′i,j of the regularization term to be added is information indicating the association between initial values of each item of the FIM. For example, it is an adjacency matrix in which “1” is set to between the initial values associated with each other and “0” is set to between the initial values not associated with each other.

An example of the adjacency matrix S′i,j included by “Ω′(W)” constituting the third regularization term in this example embodiment will be described with reference to FIG. 8. In the example of FIG. 8, the adjacency matrix S′i,j is set according to whether or not initial values in each item of the FIM are associated with each other. To be specific, in the example of FIG. 8, “L1: complete dependence on helper” and “L2: helper” as the assessment values shown in FIG. 1 are associated with each other, and “1” is set to between the initial values thereof. For example, for the item “eating”, “1” is set to “L1” and “L2” as initial values and “0” is set to “L3” as an initial value, which is true to the other items. The adjacency matrix, that is, the association between initial values is an example, and other information representing the association between initial values may be used.

Although FIG. 8 shows only part of the adjacency matrix S′i,j included by “Ω′(W)” constituting the third regularization term described above, three initial values are set for each of the eighteen items of the FIM, so that the matrix has a size of 54 columns×54 columns Along with this, unlike in the case of the first example embodiment shown in FIG. 3, the adjacency matrix Si,j included by “Ω(W)” constituting the second regularization term of this example embodiment has a size of 54 columns×54 columns as with the adjacency matrix S′i,j included by the third regularization term described above. In this case, in the adjacency matrix Si,j, the value of each element is set for each FIM item of the FIM and for each initial value as in FIG. 8. However, regardless of initial values, in consideration of only an example of association of “functions” or “categories” of the FIM items, “1” is set in a case where “functions” or “categories” of the FIM items are associated with each other, and “0” is set in the other cases. For example, for a row of the “motor” function of the FIM, “1” is set in all columns of the “motor” function regardless of the value of each initial value, and “0” is set in the other columns. Also, for a row of the “cognition” function of the FIM, “1” is set in all the columns of the “cognition” function regardless of the value of each initial value, and “0” is set in the other columns.

Thus, in this example embodiment, by adding a regularization term including an adjacency matrix corresponding to the association between initial values for each item of the FIM, it is possible to generate a model function (f_ik) corresponding to initial values of each item of the FIM associated with each other so that parameters included in the model function are similar to each other. Moreover, in this example embodiment, a regularization term including an adjacency matrix corresponding to the association between items of the FIM is disposed as in the first example embodiment, so that it is possible to generate a model function corresponding to initial values of the FIM associated with each other so that parameters included in the model function are similar to each other.

Then, the output unit 13 (predicting unit) in this example embodiment inputs patient data of a patient not discharged yet acquired as prediction data by the input unit 11 into the model function (f_ik) as described above. That is to say, the output unit 13 inputs, as an input value (X_n′), “basic information” including “gender”, “age group”, “consciousness level”, “disease name” and “paralysis condition” and “information at admission” including “initial value of each item of FIM at admission (first assessment value)” in patient data of a patient who has just been admitted into the model function, and calculates an output value (y_ik′) by the model function (f_ik(X_n′)). With this, it is possible to predict whether or not the assessment value (for example, a value of four-level scale) of each item of the FIM at the time of discharge of a patient who has just been discharged rises.

As described above, according to the present invention, a model for computing whether or not the assessment value of each item of the FIM rises is generated from information of a past rehabilitated patient in consideration of the association between the items of the FIM and the association between the initial values in each item of the FIM. Thus, it is possible to predict a change of the assessment value of each item of the FIM at the time of discharge based on the association between the items of the FIM and the association between the initial values in each item of the FIM, so that it is possible to accurately and speedily predict a change of the assessment value of each item of the FIM at the time of discharge even if an assessment index including many items is used.

Although a case of predicting a change of the assessment value of each item of the FIM at the time of discharge from patient data at the time of admission of a patient is illustrated above, it is also possible to, using patient data at any moment during hospitalization, predict a change of the assessment value of each item of the FIM at any later moment.

Further, although the assessment values of items set in the FIM are used, the values of items set in another index such that assesses the condition of a human body may be used. For example, there is an index for assessing activities of daily living such as the “Barthel Index” for assessing a total of ten items set from two viewpoints including daily living activity and locomotion activity in accordance with the degree of independence, and the values of the items of the index may be used to generate a model as described above and calculate a prediction value.

### Third Example Embodiment

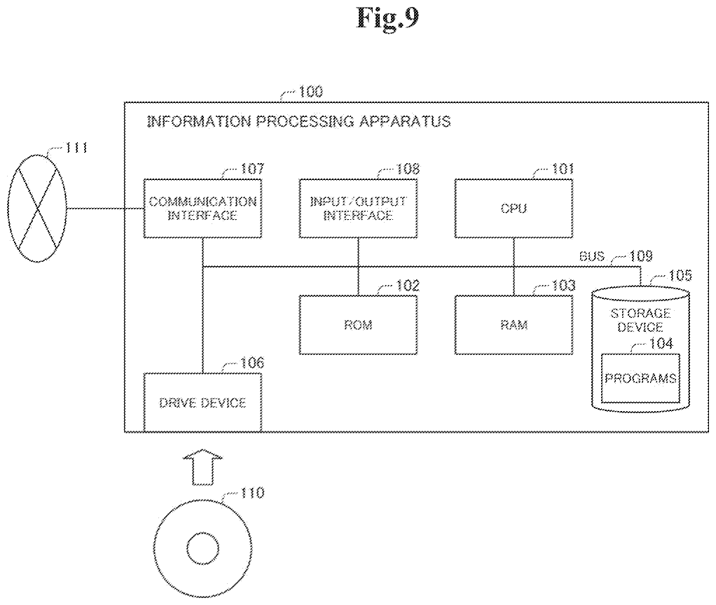

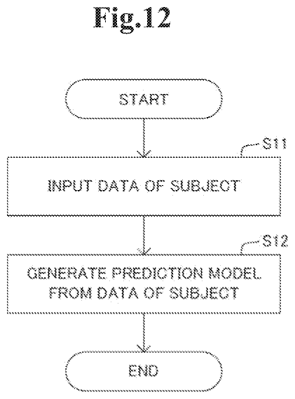

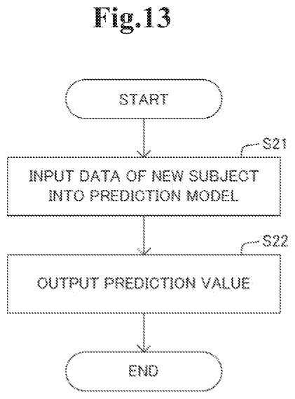

Next, a third example embodiment of the present invention will be described with reference to FIGS. 9 to 13. FIGS. 9 to 11 are block diagrams showing a configuration of an information processing apparatus in the third example embodiment, and FIGS. 12 to 13 are flowcharts showing an operation of the information processing apparatus. In this example embodiment, the overview of the configurations of the information processing apparatus and the information processing method described in the first or second example embodiment.

First, with reference to FIG. 9, a hardware configuration of an information processing apparatus 100 in this example embodiment will be described. The information processing apparatus 100 is configured by a general information processing apparatus and includes the following hardware configuration as an example:

a CPU (Central Processing Unit) 101 (arithmetic logic unit);

a ROM (Read Only Memory) 102 (storage unit);

a RAM (Random Access Memory) 103 (storage unit);

programs 104 loaded to the RAM 103;

a storage device 105 to store the programs 104;

a drive device 106 that reads from and write into a storage medium 110 outside the information processing apparatus;

a communication interface 107 connected to a communication network 111 outside the information processing apparatus;

an input/output interface 108 that inputs and outputs data; and

a bus 109 that connects the respective components.

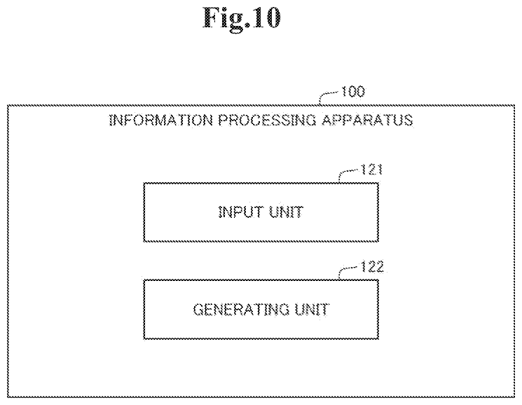

By acquisition and execution of the programs 104 by the CPU 101, the information processing apparatus 100 can structure and include an input unit 121 and a generating unit 122 shown in FIG. 10. The programs 104 are, for example, stored in the storage device 105 or the ROM 102 in advance, and loaded to the RAM 103 and executed by the CPU 101 as necessary. Moreover, the programs 104 may be supplied to the CPU 101 via the communication network 111, or may be stored in the storage medium 110 in advance to be read and supplied to the CPU 101 by the drive device 106. The abovementioned input unit 121 and generating unit 122 may be structured by an electronic circuit.

FIG. 9 shows an example of the hardware configuration of the information processing apparatus 100, and the hardware configuration of the information processing apparatus 100 is not limited to the abovementioned case. The information processing apparatus may be configured by part of the abovementioned configuration, for example, excluding the drive device 106.

The information processing apparatus 100 executes an information processing method shown in a flowchart of FIG. 12 by a function of the input unit 121 and the generating unit 122 structured by the programs as described above.

As shown in FIG. 12, the information processing apparatus 100:

accepts input of a first assessment value representing assessment of a subject at a predetermined moment and a second assessment value representing assessment of the subject after a lapse of a predetermined time from the predetermined moment for each of a plurality of items set in a FIM (Functional Independence Measure), (step S11); and

generates a model for calculating the second assessment value with respect to the first assessment value in each of the plurality of items of the FIM based on information representing an association between the items of the FIM (step S12).

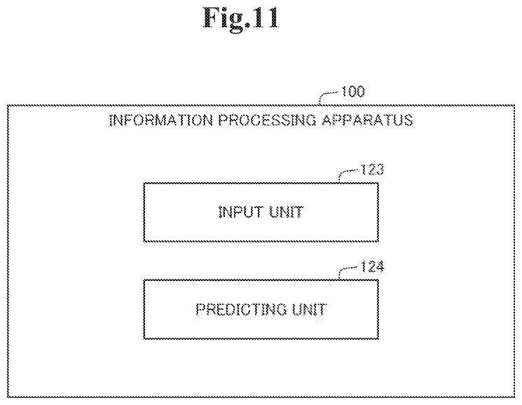

Further, by acquisition and execution of the programs 104 by the CPU 101, the information processing apparatus 100 can structure and include an input unit 123 and a predicting unit 124 shown in FIG. 11. The abovementioned input unit 123 and predicting unit 124 may be structured by an electronic circuit.

The information processing apparatus 100 executes an information processing method shown in FIG. 13 by a function of the input unit 123 and the predicting unit 124 structured by the programs as described above.

As shown in FIG. 13, the information processing apparatus 100:

inputs, into a model generated to calculate a second assessment value representing assessment of a subject after a lapse of a predetermined time from a predetermined moment with respect to a first assessment value representing assessment at the predetermined moment of the subject for each of a plurality of items set in a FIM (Functional Independence Measure) based on information representing an association between the items of the FIM, a new first assessment value for each of the plurality of items of the FIM (step S21), and outputs a value calculated with the model in accordance with the input of the new first assessment value (step S22).

The information processing apparatus 100 described above is configured by, for example, a server computer installed in a facility such as a hospital where a patient as a subject is rehabilitated, or a so-called cloud server computer operated and managed by the facility. Moreover, as described above, a value calculated and output by the information processing apparatus 100 is displayed on an information processing terminal (a personal computer, a tablet terminal, a smartphone, or the like) used by a medical professional such as a therapist or a nurse who assists the rehabilitation of a patient in the facility, and is referred to by the medical professional.

With the configuration as described above, this example embodiment generates a model for calculating an assessment value of each of items of the FIM in consideration of an association between the items of the FIM. By thus using the association between the items of the FIM, it is possible to predict an assessment value of each item of the FIM accurately and quickly even if an assessment index including many items is used. The respective example embodiments are not limited to being applied to the items set in the FIM, and can be applied to items set in an index different from the FIM for measuring the condition of a patient, and items set in any other index for assessing the condition of a human body.

### <Supplementary Notes>

The whole or part of the example embodiments disclosed above can be described as, but not limited to, the following supplementary notes.

### (Supplementary Note 1)

An information processing method comprising:

accepting input of a first assessment value and a second assessment value for each of a plurality of items set in FIM (Functional Independence Measure), the first assessment value representing assessment of a subject at a predetermined moment, the second assessment value representing assessment of the subject after a lapse of a predetermined time from the predetermined moment; and

generating a model for calculating the second assessment value with respect to the first assessment value for each of the plurality of items of the FIM based on information representing an association between the items of the FIM.

### (Supplementary Note 2)

The information processing method according to Supplementary Note 1, comprising generating the model based on information representing whether or not the items of the FIM are associated with each other.

### (Supplementary Note 3)

The information processing method according to Supplementary Note 2, comprising generating the model based on information in which the items of the FIM are associated in accordance with a content of assessment for each of the items.

### (Supplementary Note 3.1)

The information processing method according to Supplementary Note 2, comprising generating the model based on information representing whether or not the items are associated set based on a content of assessment for each of the items of the FIM.

### (Supplementary Note 4)

The information processing method according to Supplementary Note 3 or 3.1, comprising generating the model based on information in which the items of the FIM are associated in accordance with a content of assessed activity or cognition for each of the items.

### (Supplementary Note 4.1)

The information processing method according to Supplementary Note 3 or 3.1, comprising

generating the model based on information representing whether or not the items of the FIM are associated set based on a content of assessed activity or cognition for each of the items.

### (Supplementary Note 5)

The information processing method according to any of Supplementary Notes 2 to 4.1, comprising

generating the model so that parameters included by the model corresponding to the items of the FIM associated with each other become similar.

### (Supplementary Note 6)

The information processing method according to any of Supplementary Notes 2 to 5, comprising

generating the model by using a loss function to which a regularization term including an adjacency matrix representing an association between the items of the FIM is added.

### (Supplementary Note 7)

The information processing method according to any of Supplementary Notes 1 to 6, comprising

accepting the input of the first assessment value and the second assessment value for each of the plurality of items of the FIM, the first assessment value being a value representing an assessment degree of the subject at the predetermined moment, the second assessment value being a value representing an assessment degree of the subject after the lapse of the predetermined time from the predetermined moment.

### (Supplementary Note 8)

The information processing method according to any of Supplementary Notes 1 to 6, comprising

accepting the input of the first assessment value and the second assessment value for each of the plurality of items of the FIM, the first assessment value being a value representing an assessment degree of the subject at the predetermined moment, the second assessment value being a value representing whether or not an assessment degree of the subject after the lapse of the predetermined time from the predetermined moment has changed in one direction.

### (Supplementary Note 9)

The information processing method according to Supplementary Note 8, comprising:

generating the model for each of the items of the FIM and for each of the first assessment values; and also

generating a model for calculating the second assessment value with respect to the first assessment value for each of the plurality of items of the FIM based on the information representing the association between the items of the FIM and information representing an association between the first assessment values.

### (Supplementary Note 10)

The information processing method according to Supplementary Note 9, comprising

generating the model based on information representing whether or not the first assessment values are associated.

### (Supplementary Note 11)

The information processing method according to Supplementary Note 10, comprising

generating the model so that parameters included by the model corresponding to the first assessment values associated with each other become similar.

### (Supplementary Note 12)

The information processing method according to any of Supplementary Notes 9 to 11, comprising

generating the model by using a loss function to which a regularization term including an adjacency matrix representing an association between the first assessment values is added.

### (Supplementary Note 13)

The information processing method according to any of Supplementary Notes 1 to 12, comprising

inputting a new first assessment value for each of the plurality of items of the FIM into the model, and outputting a value calculated with the model in accordance with the input of the new first assessment value.

### (Supplementary Note 14)

An information processing method comprising

inputting, into a model generated to calculate a second assessment value representing assessment of a subject after a lapse of a predetermined time from a predetermined moment with respect to a first assessment value representing assessment of the subject at the predetermined moment for each of a plurality of items set in FIM (Functional Independence Measure) based on information representing an association between the items of the FIM, a new first assessment value for each of the plurality of items of the FIM, and outputting a value calculated with the model in accordance with the input of the new first assessment value.

### (Supplementary Note 15)

An information processing apparatus comprising:

an input unit configured to accept input of a first assessment value and a second assessment value for each of a plurality of items set in FIM (Functional Independence Measure), the first assessment value representing assessment of a subject at a predetermined moment, the second assessment value representing assessment of the subject after a lapse of a predetermined time from the predetermined moment; and

a generating unit configured to generate a model for calculating the second assessment value with respect to the first assessment value for each of the plurality of items of the FIM based on information representing an association between the items of the FIM.

### (Supplementary Note 16)

The information processing apparatus according to Supplementary Note 15, further comprising

a predicting unit configured to output a value calculated with the model in accordance with the input of a new first assessment value for each of the plurality of items of the FIM into the model.

### (Supplementary Note 17)

An information processing apparatus comprising:

an input unit configured to input, into a model generated to calculate a second assessment value representing assessment of a subject after a lapse of a predetermined time from a predetermined moment with respect to a first assessment value representing assessment of the subject at the predetermined moment for each of a plurality of items set in FIM (Functional Independence Measure) based on information representing an association between the items of the FIM, a new first assessment value for each of the plurality of items of the FIM; and

a predicting unit configured to output a value calculated with the model in accordance with the input of the new first assessment value.

### (Supplementary Note 18)

A computer program comprising instructions for causing an information processing apparatus to realize:

an input unit configured to accept input of a first assessment value and a second assessment value for each of a plurality of items set in FIM (Functional Independence Measure), the first assessment value representing assessment of a subject at a predetermined moment, the second assessment value representing assessment of the subject after a lapse of a predetermined time from the predetermined moment; and

a generating unit configured to generate a model for calculating the second assessment value with respect to the first assessment value for each of the plurality of items of the FIM based on information representing an association between the items of the FIM.

### (Supplementary Note 19)

The computer program according to Supplementary Note 18, comprising instructions for causing the information processing apparatus to further realize

a predicting unit configured to output a value calculated with the model in accordance with the input of a new first assessment value for each of the plurality of items of the FIM into the model.

### (Supplementary Note 20)

A computer program comprising instructions for causing an information processing apparatus to realize:

an input unit configured to input, into a model generated to calculate a second assessment value representing assessment of a subject after a lapse of a predetermined time from a predetermined moment with respect to a first assessment value representing assessment of the subject at the predetermined moment for each of a plurality of items set in FIM (Functional Independence Measure) based on information representing an association between the items of the FIM, a new first assessment value for each of the plurality of items of the FIM; and

a predicting unit configured to output a value calculated with the model in accordance with the input of the new first assessment value.

### (Supplementary Note 21)

An information processing method comprising:

accepting input of a first assessment value and a second assessment value for each of a plurality of items set in a predetermined index for assessing a human body, the first assessment value representing assessment of a subject at a predetermined moment, the second assessment value representing assessment of the subject after a lapse of a predetermined time from the predetermined moment; and

generating a model for calculating the second assessment value with respect to the first assessment value for each of the plurality of items of the predetermined index based on information representing an association between the items of the predetermined index.

### (Supplementary Note 22)

The information processing method according to Supplementary Note 21, comprising

inputting a new first assessment value for each of the plurality of items of the predetermined index into the model, and outputting a value calculated with the model in accordance with the input of the new first assessment value.

### (Supplementary Note 23)

An information processing method comprising

inputting, into a model generated to calculate a second assessment value representing assessment of a subject after a lapse of a predetermined time from a predetermined moment with respect to a first assessment value representing assessment of the subject at the predetermined moment for each of a plurality of items set in a predetermined index for assessing a condition of a human body based on information representing an association between the items of the predetermined index, a new first assessment value for each of the plurality of items of the predetermined index, and outputting a value calculated with the model in accordance with the input of the new first assessment value.

The abovementioned program can be stored using various types of non-transitory computer-readable mediums and supplied to a computer. The non-transitory computer-readable mediums include various types of tangible storage mediums. Examples of the non-transitory computer-readable mediums include a magnetic recording medium (for example, a flexible disk, a magnetic tape, a hard disk drive), a magnetooptical recording medium (for example, a magnetooptical disk), a CD-ROM (Read Only Memory), a CD-R, a CD-R/W, and a semiconductor memory (for example, a mask ROM, a PROM (Programmable ROM), an EPROM (Erasable PROM) a flash ROM, a RAM (Random Access Memory)). Moreover, the program may be supplied to a computer by various types of transitory computer-readable mediums. Examples of the transitory computer-readable mediums include an electric signal, an optical signal, and an electromagnetic wave. The transitory computer-readable mediums can supply the program to a computer via a wired communication path such as a wire and an optical fiber or a wireless communication path.

Although the present invention has been described with reference to the above example embodiments and so on, the present invention is not limited to the example embodiments. The configurations and details of the present invention can be changed in various manners that can be understood by one skilled in the art within the scope of the present invention.

### DESCRIPTION OF NUMERALS

10 information processing apparatus
11 input unit
12 learning unit
13 output unit
14 data storing unit
15 model storing unit
20 data management apparatus
100 information processing apparatus
101 CPU
102 ROM
103 RAM
104 programs
105 storage device
106 drive device
107 communication interface
108 input/output interface
109 bus
110 storage medium
111 communication network
121 input unit
122 generating unit
123 input unit
124 predicting unit

What is claimed is:

1. An information processing method comprising:
accepting input of a first assessment value and a second assessment value for each of a plurality of items set in FIM (Functional Independence Measure), the first assessment value representing assessment of a subject at a predetermined moment, the second assessment value representing assessment of the subject after a lapse of a predetermined time from the predetermined moment; and
generating a model for calculating the second assessment value with respect to the first assessment value for each of the plurality of items of the FIM based on information representing an association between the items of the FIM.

2. The information processing method according to claim 1, comprising
generating the model based on information representing whether or not the items of the FIM are associated with each other.

3. The information processing method according to claim 1, comprising
generating the model based on information in which the items of the FIM are associated in accordance with a content of assessment for each of the items.

4. The information processing method according to claim 3, comprising
generating the model based on information in which the items of the FIM are associated in accordance with a content of assessed activity or cognition for each of the items.

5. The information processing method according to claim 2, comprising
generating the model so that parameters included by the model corresponding to the items of the FIM associated with each other become similar.

6. The information processing method according to claim 2, comprising
generating the model by using a loss function to which a regularization term including an adjacency matrix representing an association between the items of the FIM is added.

7. The information processing method according to claim 1, comprising
accepting the input of the first assessment value and the second assessment value for each of the plurality of items of the FIM, the first assessment value being a value representing an assessment degree of the subject at the predetermined moment, the second assessment value being a value representing an assessment degree of the subject after the lapse of the predetermined time from the predetermined moment.

8. The information processing method according to claim 1, comprising
accepting the input of the first assessment value and the second assessment value for each of the plurality of items of the FIM, the first assessment value being a value representing an assessment degree of the subject at the predetermined moment, the second assessment value being a value representing whether or not an assessment degree of the subject after the lapse of the predetermined time from the predetermined moment has changed in one direction.

9. The information processing method according to claim 8, comprising:
generating the model for each of the items of the FIM and for each of the first assessment values; and also
generating a model for calculating the second assessment value with respect to the first assessment value for each of the plurality of items of the FIM based on the information representing the association between the items of the FIM and information representing an association between the first assessment values.

10. The information processing method according to claim 9, comprising
generating the model based on information representing whether or not the first assessment values are associated.

11. The information processing method according to claim 10, comprising
generating the model so that parameters included by the model corresponding to the first assessment values associated with each other become similar.

12. The information processing method according to claim 9, comprising
generating the model by using a loss function to which a regularization term including an adjacency matrix representing an association between the first assessment values is added.

13. The information processing method according to claim 1, comprising
inputting a new first assessment value for each of the plurality of items of the FIM into the model, and outputting a value calculated with the model in accordance with the input of the new first assessment value.

14. (canceled)

15. An information processing apparatus comprising:
at least one memory configured to store processing instructions; and
at least one processor configured to execute processing instructions to:
accept input of a first assessment value and a second assessment value for each of a plurality of items set in FIM (Functional Independence Measure), the first assessment value representing assessment of a subject at a predetermined moment, the second assessment value representing assessment of the subject after a lapse of a predetermined time from the predetermined moment; and
generate a model for calculating the second assessment value with respect to the first assessment value for each of the plurality of items of the FIM based on information representing an association between the items of the FIM.

16. The information processing apparatus according to claim 15, wherein the at least one processor is configured to execute processing instructions to
output a value calculated with the model in accordance with the input of a new first assessment value for each of the plurality of items of the FIM into the model.

17. (canceled)

18. A non-transitory computer-readable storage medium having a computer program stored therein, the computer program comprising instructions for causing an information processing apparatus to execute processing to:
accept input of a first assessment value and a second assessment value for each of a plurality of items set in FIM (Functional Independence Measure), the first assessment value representing assessment of a subject at a predetermined moment, the second assessment value representing assessment of the subject after a lapse of a predetermined time from the predetermined moment; and
generate a model for calculating the second assessment value with respect to the first assessment value for each of the plurality of items of the FIM based on information representing an association between the items of the FIM.

19. The non-transitory computer readable storage medium having the computer program stored therein according to claim 18, the computer program comprising instructions for causing the information processing apparatus to further execute processing to
output a value calculated with the model in accordance with the input of a new first assessment value for each of the plurality of items of the FIM into the model.

20. (canceled)
